(19) United States
(12) Patent Application Publication
(10) Pub. No.: US 2026/0144955 A1
(43) Pub. Date: May 28, 2026

(54) Audio Therapy

(71) Applicant: Stress Point Health Limited, London (GB)

(72) Inventors: Sheena Pirbhai, London (GB); Daniel Frederik Maria Stevens, London (GB)

(73) Assignee: Stress Point Health Limited, London (GB)

(21) Appl. No.: 19/266,383

(22) Filed: Jul. 11, 2025

Related U.S. Application Data
(63) Continuation of application No. PCT/GB2024/050082, filed on Jan. 12, 2024.

(30) Foreign Application Priority Data
Jan. 12, 2023 (GB) 2300478.1

(51) Int. Cl. A61M 21/00; G16H 20/30

(52) U.S. Cl. CPC A61M 21/00; G16H 20/30; A61M 2021/0027

(57) ABSTRACT

A computer implemented method for generating digital audio signals for stimulating a user's brain, comprising the steps of: generating a first audio signal which changes frequency at a first rate for playback to a patient through a first speaker of a stereo headset; receiving an input which identifies a stressor frequency for the patient; generating a second audio signal which changes frequency at a second rate for playback to the patient through a second speaker of the stereo headset, while simultaneously playing an audio signal at the stressor frequency through the first speaker of the stereo headset, wherein an initial frequency for the second audio signal is selected to be within a range extending from the stressor frequency to a frequency 40 Hz higher than the stressor frequency; receiving an input which identifies a disruptor frequency for the user; and, storing the identified stressor frequency and the identified disruptor frequency for subsequent playback to the user.

### BACKGROUND

Neuroacoustic feedback (NAF) refers to a type of vibroacoustic therapy characterised by stimulating a patient's brain, including harmonising brain wave activity already present in the brain of a patient. Various mental health disorders are underpinned by emotional dysregulation and damaged brain patterns, where patients have a diminished capacity to effectively manage and control their reactions to an emotional and/or traumatic experience. NAF can help improve cognitive abilities, such as memory and attention, and can also have a positive effect on sensory processing, such as reducing sensitivity to certain sounds or improving the ability to process and interpret auditory information. NAF is often used as a treatment for conditions such as mental health conditions that may result from traumatic experiences, autism, and ADHD.

Research shows that the frequencies of electrical activity or neural discharge patterns of the brain correspond to certain mental, emotion and cognitive states. The emotional regulation centre or anterior cingulate cortex area of the brain processes emotions. With emotional dysregulation this area of the brain is under active and can therefore lead to extreme emotional responses to triggers or lack of emotional feeling at all. By matching the ‘damaged’ brain wave frequency and inducing a new frequency, this can make the damaged brain pattern healthier such that the patient does not have to suffer from the stress trigger again.

Traditionally, the audio signals for use in NAF are provided through an analogue device such as a BioAcoustical Utilization Device (BAUD). BAUD type devices use specific frequencies and intensities of sound to create vibrations that are applied to the human body, either through speakers or other devices. The vibrations produced by a BAUD device may help to create healthy brain patterns, which can reduce stress and improve overall well-being. A typical BAUD device has four dials, two of which can be adjusted manually to control the frequency of the sound signals, allowing the patient to tailor the therapy to their specific needs. A BAUD device uses gradients of frequencies and differential stimulation between two speakers to produce resonance for therapeutic impact.

In BAUD type devices, the frequencies used by the patient are selected using two dials. Each dial is manually turned by the user in order to identify the frequencies for use with the neuroacoustic feedback therapy. This can make it challenging for a user to determine the required frequencies, which can be determinantal to (or even entirely prevent) the effectiveness of the NAF treatment.

### SUMMARY OF THE INVENTION

According to one aspect, there is provided a computer implemented method for generating digital audio signals for stimulating a user's brain, comprising the steps of: generating a first audio signal which changes frequency at a first rate for playback to a patient through a first speaker of a stereo headset; receiving an input which identifies a stressor frequency for the patient; generating a second audio signal which changes frequency at a second rate for playback to the patient through a second speaker of the stereo headset, while simultaneously playing an audio signal at the stressor frequency through the first speaker of the stereo headset, wherein an initial frequency for the second audio signal is selected to be within a range extending from the stressor frequency to a frequency 40 Hz higher than the stressor frequency; receiving an input which identifies a disruptor frequency for the user; and storing the identified stressor frequency and the identified disruptor frequency for subsequent playback to the user.

In an embodiment, there is provided a method wherein a frequency range of the first audio signal is between 40 Hz and 1.5 kHz.

In another embodiment, there is provided a method wherein an initial frequency for the first audio signal is at least 40 Hz and is thereafter increased incrementally at the first rate.

In another embodiment, there is provided a method wherein an initial frequency for the first audio signal is determined using user data.

In another embodiment, there is provided a method wherein an initial frequency for the second audio signal is selected to be equal to the stressor frequency and is thereafter increased incrementally at the second rate.

In another embodiment, there is provided a method wherein the input which identifies the stressor frequency and/or the disruptor frequency is a user input, preferably a touch gesture.

In another embodiment, there is provided a method wherein the input which identifies the stressor frequency and/or the disruptor frequency is a user biomarker signal.

In another embodiment, there is provided a method in which the user biomarker signal is monitored heart rate variability (HRV).

In another embodiment, there is provided a method wherein the stressor frequency is determined by a reduction in an HRV value for the user, and the disruptor frequency is determined by an increase in an HRV value for the user.

In another embodiment, there is provided a method wherein the first rate is higher than the second rate.

In another embodiment, there is provided a method wherein the first rate gives rise to a non-linear incremental change in frequency of the first audio signal.

In another embodiment, there is provided a method in which the first rate increases the frequency of the first audio signal in increments of k×f_{c}, where f_{c} is the current frequency of the first audio signal and k is a constant.

In another embodiment, there is provided a method wherein k is a value of at least 1, and preferably at least 1.03.

In another embodiment, there is provided a method wherein the second rate gives rise to linear incremental change in frequency of the second audio signal.

In another embodiment, there is provided a method in which the second rate increases the frequency of the second audio signal in increments of at least 0.2 Hz per second, and preferably at least 0.5 Hz per second.

In another embodiment, there is provided a method in which the first audio signal and the second audio signal are simple sine waves with minimal coloration.

In another embodiment, there is provided a method wherein the headset comprises a set of over-ear headphones, on-ear headphones, in-ear speaker buds, or bone conduction speakers.

In another embodiment, there is provided a method wherein the first audio signal and the second audio signal are not pre-recorded.

According to another aspect, there is provided a computer program comprising instructions, that when executed, perform a method described in above aspects or embodiments.

According to another aspect, there is provided a digital audio processing device including instructions, that when executed by a processor, are configured to cause the processor to perform the method described in above aspects or embodiments.

In an embodiment, there is provided device comprising a smartphone, a smart watch, a tablet computer, a laptop computer, or a personal wellness pod.

According to another aspect, there is provided a method of treatment for a health condition using neuroacoustic feedback (NAF) therapy, comprising the steps of generating a first audio signal at a stressor frequency and a second audio signal at a disruptor frequency for simultaneous playback to a user through respective speakers of a stereo headset, wherein the stressor frequency and the disruptor frequency are determined by the method described in above aspects or embodiments.

In an embodiment, there is provided method of treatment wherein the health condition is one of stress, anxiety, post-traumatic stress disorder (PTSD), depression, addiction, attention deficit hyperactivity disorder (ADHD), bipolar affective disorder, phobia, behavioural and emotional disorders with children, eating disorders, autism and obsessive-compulsive disorder (OCD).

In another embodiment, there is provided method of treatment further comprising administering a pharmaceutical treatment as a co-therapy for treating the health condition.

### BRIEF DESCRIPTION OF THE DRAWINGS

One or more examples of the present disclosure will now be described in detail with reference to the accompanying drawings, in which:

FIG. 1 shows a flow chart illustrating a method for generating digital audio signals according to the present disclosure;

FIG. 2 illustrates a headset emitting a stressor frequency according to the present disclosure;

FIG. 3 illustrates a headset emitting a disruptor frequency according to the present disclosure;

FIG. 4 illustrates a headset emitting a stressor frequency and a disruptor frequency, which combine to form a beating frequency according to the present disclosure;

FIG. 5 shows an example of a system for implementing a method for generating digital audio signals according to the present disclosure;

FIG. 6 illustrated features of a user device for use in implementing a method for generating digital audio signals according to the present disclosure; and,

FIG. 7 illustrates the use of a graphical user interface (GUI) in a system according to the present disclosure.

### DETAILED DESCRIPTION

According to the present disclosure, there is provided a computer implemented method for generating digital audio signals suitable for stimulating a user's brain. In particular, there is provided an improved method for identifying and selecting a stressor frequency and a corresponding disruptor frequency, for use in treatment and therapy protocols involving neuroacoustic feedback (NAF).

There is also provided a digital audio processing device suitable for implementing the method, and a method of treatment incorporating the use of audio signals selected and generated according to the method.

As discussed in more detail below, audio signals generated according to the present disclosure may be used in order to help treat various neurological conditions including (but not limited to) stress, anxiety, post-traumatic stress disorder (PTSD), depression, addiction, attention deficit hyperactivity disorder (ADHD), bipolar affective disorder, phobia, behavioural and emotional disorders with children, eating disorders and obsessive-compulsive disorder (OCD).

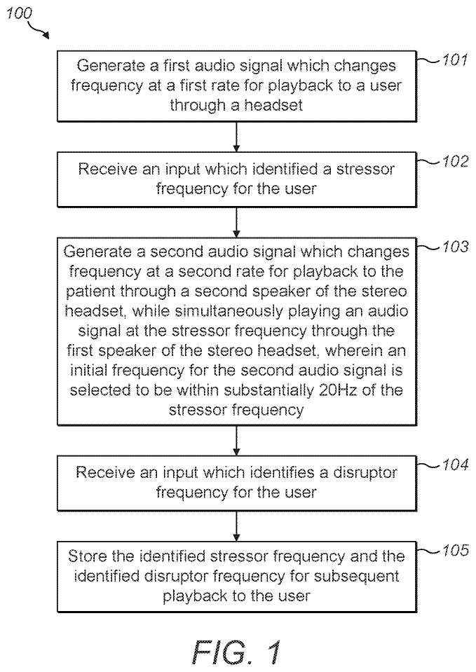

FIG. 1 illustrates a flow chart showing a method for generating digital audio signals for stimulating a user's brain, according to the present disclosure. In a first step 101, a first audio signal is generated for playback to a user through a headset 600. The first audio signal, when played through a headset 600, is audible to the user. The frequency of the first audio signal, when played through a headset, may be referred to as a “sound frequency” or alternatively as a “tone frequency”. The frequency of the first audio signal is typically measured in Hertz (Hz). In some examples, the frequency of the first signal is between 40 Hz and 1500 Hz (1.5 kHz).

The frequency of the first audio signal is then changed at a first rate. In some examples, the frequency of the first audio signal is initiated at a starting frequency. In some examples, the starting frequency is 40 Hz and the frequency of the first audio signal is thereafter increased incrementally at the first rate. In some examples, the starting frequency may start at a pre-determined frequency between 40 Hz and 1500 Hz and is thereafter increased incrementally at the first rate. As discussed in more detail below, the starting frequency for the first audio signal may also be determined based on input data 300. Such input data 300 may include, for example, user data. In such examples, once a starting frequency has been determined, the frequency is set to the starting frequency and is thereafter increased incrementally at the first rate.

After a starting frequency has been determined (or selected, pre-determined etc.) the first audio signal is played to the user through a headset 600. In some examples, the first audio signal is played to the user through only one side of the headset 600, such that the user hears the first audio signal through only one ear.

The frequency of the first audio signal is then increased incrementally, while the user listens to the playback of the first audio signal. In some examples, the first rate is a pre-determined rate. In some examples, the first rate is such that the incremental increase in the frequency of the first audio signal is non-linear. In some examples, the incremental change in frequency according to the first rate is determined using the following equation:

$${f}_{n} = k \times {f}_{c}$$

Where fₙ is the frequency of the next increment, f_{c} is the current frequency and k is a constant. In some examples, the value of k is at least 1 and may be equal to 1.03.

In a second step 102, while the user is listening to the first audio signal through a headset 600, an input identifies a stressor frequency for the user. In some examples, such an input indicates that the first audio signal is presently at a tone frequency that is perceived by the user as inducing a heightened stress state in the user. In some examples, the input to identify the stressor frequency may be a user input provided by the user, for example using a touch gesture input into a user device 500.

The stressor frequency may vary depending on various factors, including (but not limited to) the physical characteristics of the user, for example biomarkers such as blood pressure or physical health, the mental condition of the user, and the type of neurological disorder for which treatment is sought. The stressor frequency may also be different for different sessions conducted by the same user in order to treat the same neurological condition.

In a third step 103, after the input has been received by the user identifying the stressor frequency, a second audio signal is generated. The starting frequency of the second audio signal is selected to be within a range extending from the identified stressor frequency to a frequency 40 Hz higher than the identified stressor frequency. In an example, the starting frequency of the second audio signal is equal to the stressor frequency.

The frequency of the second audio signal is then increased incrementally within the range, while the user listens to the playback of the second audio signal through a headset 600. The second audio signal is played only through one side of the headset, while the stressor signal is played only through the other side of the headset. As such, the user will hear the stressor signal through one ear, while hearing the second audio signal through the other ear.

Beginning from the starting frequency of the second audio signal, the frequency of the second audio signal is incrementally increased at a second rate. In some examples, the second rate is lower than the first rate. In some examples, the second rate is such that the incremental increase in the frequency of the second audio signal is linear. In some examples, the second rate is such that the frequency of the second audio signal increases in increments of at least 0.2 Hz per second (Hz/s), and in some examples is at least 0.5 Hz/s.

In a fourth step 104, while the user is listening to the stressor signal and the second audio signal through a headset 600, an input identifies a disruptor frequency for the user. In some examples, such an input indicates that the second audio signal is presently at a tone frequency that is perceived by the user as reducing stress to the user. In some examples, the input to identify the disruptor frequency may be a user input provided by the user. In some examples, the user input may be indicated using a touch gesture input into a user device 500.

In some examples, the disruptor frequency is within a range extending from the identified stressor frequency to 40 Hz above the identified stressor frequency.

In a fifth step 105, the identified stressor frequency and the identified disruptor frequency are stored for use in subsequent playback to the user in a therapeutic treatment. Storage of the identified stressor and disruptor frequencies may be achieved through any suitable means. For example, the frequency (in Hz) of each of the stressor and disruptor frequencies may be stored in a computer memory.

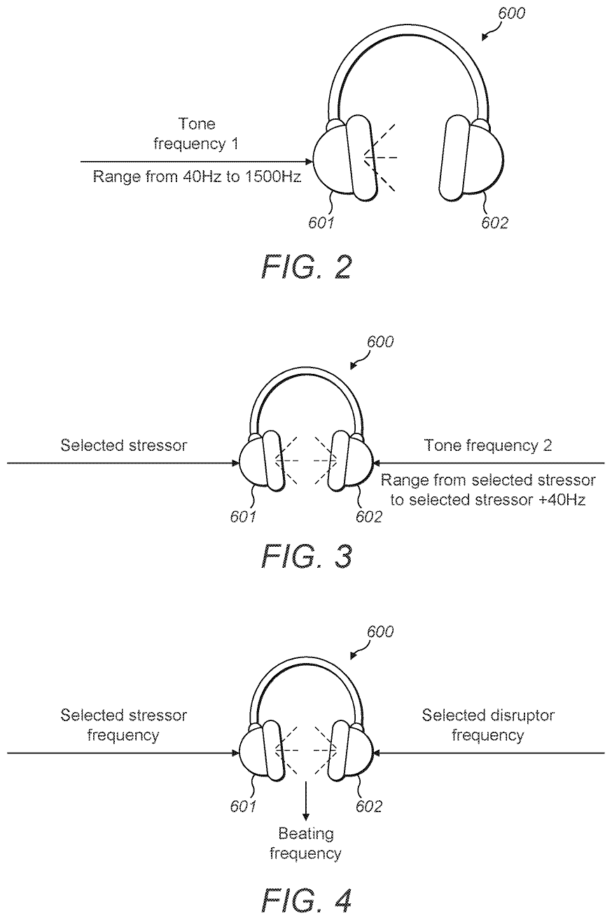

The first audio signal and the second audio signal are played to the user through a stereo headset 600, as illustrated in FIGS. 2 to 4. The first side 601 and second side 602 of the stereo headset 600 may also be referred to as the first speaker 601 and second speaker 602 of the stereo headset 600. A suitable stereo headset 600 may be over-ear headphones, on-ear headphones, in-ear speaker buds or bone conduction speakers.

Consistent with the known NAF therapies, before the first step 101 is undertaken, the user first focuses on stress inducing or triggering thoughts in order to induce a heightened stress state. Such stress inducing thoughts may be, for example, thoughts relating to the neurological condition for which treatment is sought. In examples where the user first focuses on stress inducing thoughts, the user is thus in a heightened stress state when the first step 101 is started. In such examples, the stressor frequency may induce an even higher stress state in the user.

In FIG. 2, a first tone frequency is shown being played through the first side 601 of the headset 600. In this example, the first tone frequency is the first audio frequency discussed above and is being incrementally increased through a range extending from 40 Hz to 1500 Hz, such that the stressor frequency of the user can be identified.

In FIG. 3, an input has been received identifying the stressor frequency, which is now being played through the first side 601 of the headset 600. The second tone frequency is shown being played through the second side 602 of the headset 600. In this example, the second tone frequency is the second audio frequency discussed above and is incrementally increased through a range extending from the stressor frequency to a frequency 40 Hz above the stressor frequency, such that the disrupter frequency of the user can be identified.

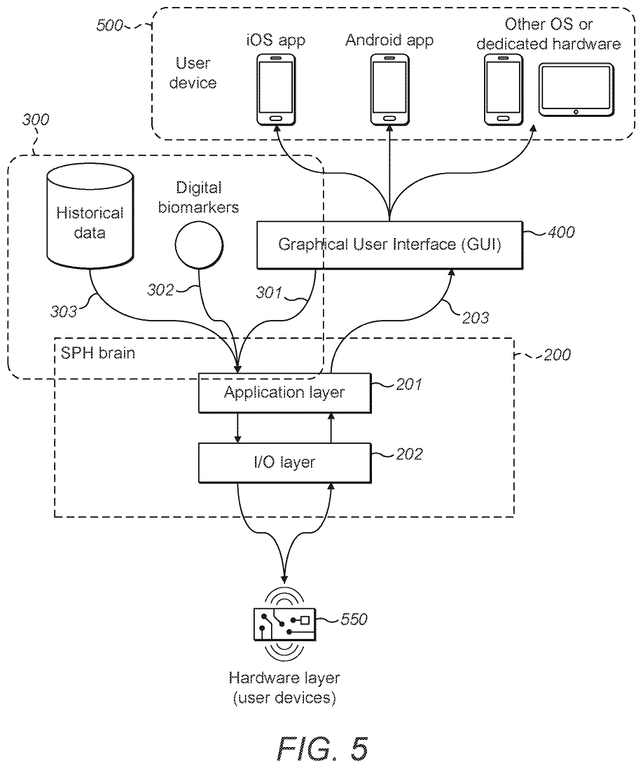

In FIG. 4, an input has been received identifying the disruptor frequency. The stressor frequency is being played only through the first side 601 of the headset 600, while the disruptor frequency is being played only through the second side 602 of the headset 600. The stressor frequency and the disruptor frequency combine to produce a beating frequency. In acoustics, a beat is an interference pattern between two sounds of slightly different frequencies, perceived as a periodic variation in volume whose rate is the difference of the two frequencies. The beating frequency is the rhythmic pattern perceived by the user as a result of the combination between the stressor frequency and the disruptor frequency.

Treatment and therapy protocols involving NAF typically work by making use of the beating frequency, as produced by the combination of the determined stressor and disruptor frequencies. In a typical treatment session, the user will hear the beating frequency for around 20 minutes. Each time the user undertakes a NAF treatment/therapy session, they will need to undertake the method outlined in FIG. 1 to set the correct frequencies.

In some examples, the input received in the second step 102 to identify the stressor frequency and/or the input received in the fourth step 104 to identify the disruptor frequency may be a user biomarker signal. The user biomarker signal may allow for automatic determination of when the stressor signal and/or disruptor signal have been reached, by monitoring one or more physical characteristics of the user.

In some examples, the user biomarker signal is monitored heart rate variability (HRV). In such examples, the user is monitored by one or more devices in order to determine HRV. HRV is the physiological phenomenon of variation in the time interval between heartbeats. HRV can be used as a psychological stress indicator. Suitable devices for monitoring HRV may be, for example: a light sensor on a smartphone, a smart watch, a smart ring, a heart rate monitoring device, a smart bandage. In some examples, one or more of these devices may be connected to an application configured to implement the method illustrated in FIG. 1, such that the HRV information can be used to determine the stressor frequency and/or the disruptor frequency. In some examples, the application is configured to monitor HRV while the stressor frequency and the disruptor frequency are being determined. Any changes in the HRV are registered and associated with the frequency being experienced by the user at the time. In some examples, this can enable the application to autonomously set the correct stressor and/or disruptor frequencies for the user. Such a method may allow for a more accurate and precise determination of the stressor and/or disruptor frequencies than could be achieved by the user indicating the frequencies manually through a touch gesture. This may, for example, be because monitoring HRV can allow the stressor and/or disruptor frequencies to be determined based on the physical reaction of the user to stress (for example, by triggering a direct response from the protection mechanism of the user's body) instead of being determined by an emotional reaction of the user, which may be delayed.

In some examples, the heart rate (HR) and HRV are monitored to help determine the stressor and/or disruptor frequencies. As the frequency of the first audio signal is increased at a first rate, the frequency first audio signal will get closer to the stressor frequency, and the stress level of the user will increase. As stress increases, the user's HR will increase. When the point of maximum stress has been encountered, the HRV value will trend towards zero, since the stress levels of the patient are no longer measurably increasing. Thus, when increasing the frequency of the first audio signal, the stressor frequency can be determined by identifying the frequency at which a reduction in the HRV is observed. In some examples, the stressor frequency may be determined by identifying the frequency of the first audio signal at which the HRV is at a minimum.

When determining the disruptor frequency, the stressor frequency is played to the user through a stereo headset 600. Thus, at the beginning of the process for determining the disruptor frequency, the user is in a state of elevated stress (due to the stressor frequency). As the frequency of the second audio signal is increased at a second rate, the frequency of the second audio signal should approach the disruptor frequency. In examples where the heart rate (HR) and HRV are monitored, this should be observed by a reduction in the heart rate, and thus the observed HRV will trend away from zero. Thus, when increasing the frequency of the second audio signal, the disruptor frequency can be determined by identifying a frequency of the second audio signal at which the HRV trends away from zero. In some examples, the disruptor frequency may be determined by identifying the frequency of the second audio signal at which the HRV is no longer changing. In some examples, when the HRV has not changed during a pre-determined time period, it is defined as no longer changing. In some examples, the period for determining that the HRV has not changed may be, for example, 1 minute.

FIG. 5 illustrates an example of a system for use in implementing the method illustrated in FIG. 1. An application 200 includes instructions that, when executed, perform the method. In the illustrated example, the application 200 is referred to as the “SPH Brain”. The application 200 is a digital engine that can be connected to a frontend such that it can be used on a user device 500 by a user. In some examples, the frontend may be a graphical user interface (GUI) 400 configured to run on a user device 500 in order to allow the application to be used by a user.

In some examples, the application 200 may be an application programming interface (API) which can be used by other applications for use in therapies employing neuroacoustic feedback. The application can be used as part of a treatment and/or therapy for a broad range of mental health issues, including stress, anxiety, post-traumatic stress disorder (PTSD), depression, addiction, attention deficit hyperactivity disorder (ADHD), bipolar affective disorder, phobia, behavioural and emotional disorders with children, eating disorders and obsessive-compulsive disorder (OCD).

In some example implementations, the application 200 may act as the engine between the GUI 400 and the hardware 550 of the user device 500. In such implementations, the application 200 includes an application layer 201 and an I/O layer 202. The application layer 201 receives input data 300. The input data 300 may include user input 301 from the GUI 400 and may include additional input from other sources such as from digital biomarkers 302 and historical data 303. In some examples, the application layer 201 processes input data 300 in order to identify potential frequency ranges for use in identifying the stressor frequency and/or the disruptor frequency. The application layer then instructs the I/O layer 202 to interact with the hardware layer 550 of the user device in order to produce the required audio frequencies and may also pass information back to the GUI 400 for presentation to the user.

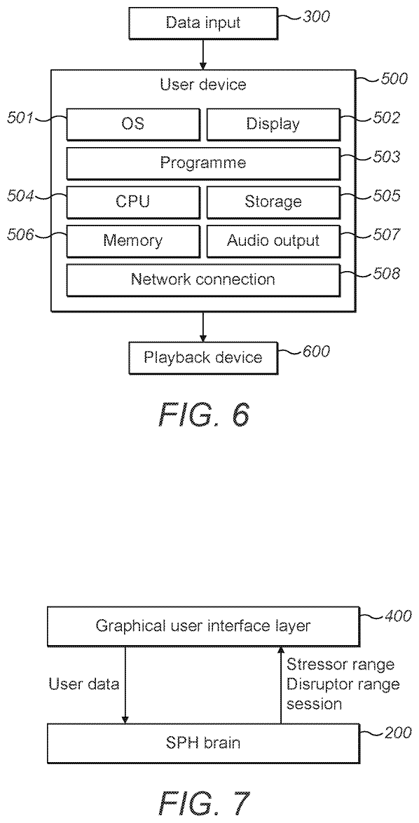

FIG. 6 illustrates an example of a user device 500 that may be used in order to implement the method of the present disclosure. The user device 500 may be any suitable device with which the user can interact with a GUI 400 configured to work with the application 200. In some examples, the user device 500 may be a smart phone, a tablet, a smart watch, a desktop computer, a laptop computer, and a personal wellness pod.

A user device 500, as illustrated in FIG. 6, may include an operating system (OS) 501 configured to support usage of the application 200. The user device 500 may also include a display 502 configured to present information to the user in visual and/or tactile form. The display 502 may also be configured to accept user input, in order for the user to interact with the user device 500. The user device 500 may also include a programme 503, which includes the application 200 and a GUI 400. The application 503 may, for example, be an app that is downloadable from an app store or from the internet. The user device 500 further includes a central processing unit (CPU) 504 configured to execute instructions in order to run the application 503. The user device 500 further includes memory 506, which may be rapid access memory (RAM) configured to provide the CPU 504 with rapid access to information. The user device 500 further includes storage 505 configured to store information accessible by the user device. The storage 505 may be contained within the user device 505 or may be external to the user device 500. In some examples, the storage 505 may be located remotely (for example in the cloud) and accessible by the user device 500 using a network and/or internet connection 508. The user device 500 is configured to receive input data 300 and to output an audio signal to a playback device, such as a headset 600.

The GUI 400 is the visual interface that is located between the application 200 and the user. In some examples, the GUI 400 may be developed by third parties who want to implement the application 200 in an alternative system. The GUI 400 collects the required information from the user and passes it through to the application 200. FIG. 7 illustrates example interactions between the application 200 and the GUI 400, when running on a user device 500.

In some examples, the application 200 may be provided with input data 300. The input data may include user input 301, digital biomarkers 302 and/or historical data 303. In some examples, the application 200 may use some or all of this input data in order to calculate pre-determined ranges for the stressor frequency and/or the disruptor frequency. In determining these frequency ranges, the application 200 may reduce the range of frequencies for the user to test in order for the stressor and/or disruptor frequencies to be determined. This could be beneficial in reducing the amount of time needed to identify the frequencies.

User input 301 may contain personal information relating to the user. This information may include intake data (country, email address, gender and date of birth), mental health details (reason for using the application and symptoms), current mental state details (anxiety scale, patient global impression of change (PGIC) scale, selected stressor frequency and selected disruptor frequency).

Historical data 303 may contain the results of previous analysis performed by an application 200 in relation to the user, or to other users of the application 200. This may include data analysed by application 200 applications running on other user devices 500. By combining user specific data relating to the present user with historical data (which may include data relating to other users), the application 200 may be able to determine more accurately the target ranges to be used by the user when determining the stressor and/or disruptor frequencies in any given session.

Digital biomarkers 202 (also referred to as user biomarkers) may include information regarding the physical characteristics of the user. In some examples, the digital biomarkers 202 may include heart rate information. Digital biomarkers 202 may include information provided by one or more external devices, including (but not limited to) the light sensor on a smartphone, a smart watch, a smart ring, a heart rate monitoring device, and a smart bandage. In some examples, the digital biomarkers 202 are provided to the application 200 by one or more external devices by using third party API integrations.

In some examples, the application 200 may predict the number of neuroacoustic feedback therapy/treatment sessions that may be required by the user, and the interval between sessions, by using third party passive prediction of user mental health state services. These are not visible to the user and are passive and automatic processes. User information is automatically collected and is used by the application 200 to deliver personal, relevant interventions when they need them with no delay.

The application 200 may collect data on the way the user interacts with their user device 500 and external biomarker details 202. This may occur on a daily basis, even when the user is not directly using the application 200. The application 200 may use this gathered background information to build a mental health state prediction model of the user. Based on the score determined by the application 200, the application 200 can alert the user to conduct another treatment/therapy session. An example of such background usage data that may be collected by the application is the number of times a user puts their user device 500 into a standby mode, how much screen time they had and during what times of the day. Also, variation in digital biomarkers (for example, heart rate) can be used by the application 200 to feed the prediction model.

In an example, the application 200 may be configured to assist the user in determining the stressor frequency and/or the disruptor frequency. In an example, the application 200 may use the following steps to assist the user:
Step 1—Based on user data (such as gender, age and the neurological/mental health condition for which the usage of the application is intended to help treat) the application will place the user in one of several predetermined frequency brackets. Such frequency brackets will be quite generic and broad.
Step 2—Based on symptoms selected by the user (for example, using the GUI) the predetermined frequency range can then be narrowed. By combining the broad frequency bracket identified in step 1, with information determined in step 2, a narrower and more effective frequency range can be determined.
Step 3—Biomarker data, such as HRV data, is input to the application 200 in the form of a digital biomarker 302 (for example, via the API) and is used to direct the user towards the correct frequency range, based on the range identified in steps 1 and 2. In this way the application 200 can predict in which frequency range it expects the stressor and/or disruptor frequency of the user to be located.

Steps 1 and 2 may incorporate historical data collected from many users of the application 200, which may help to improve the accuracy of the predictions made by the application 200 over time.

In some examples, the GUI 400 may be developed using a cross-platform software for creating GUIs. In some examples, the cross-platform software may use a QT development framework and/or a Flutter development framework.

In some examples, the signals generated for use as the first audio signal and the second audio signal (and thus, for the stressor frequency and the disruptor frequency) are generated as needed, instead of being pre-recorded. This may be advantageous, for example by reducing the storage size and/or internet usage of the application 200, since pre-recorded tones do not need to be stored and/or streamed from a remote source. This may also be advantageous in allowing for the application 200 to operate in circumstances where an internet connection may not be available.

In some examples, a development framework may be used in order to allow for the application 200 to generate the required audio signals. In some examples, JUCE may be used. JUCE is an open-source cross-platform C++ application framework, used for the development of desktop and mobile applications. In some examples, using a suitable development framework (for example, JUCE) can allow the application 200 to communicate with the hardware layer 550 of a user device 500. In such examples, the application 200 can interact with an audio module of the user device 500 in order to generate the required audio signals, thereby preventing the need for pre-recorded tones to be stored and/or streamed by the application.

In some examples, user and/or application data may be stored in a remote cloud-based storage system. In some examples, such a storage system may be a backup of information stored locally on the user device 500. Any suitable storage environment may be used, in some examples the storage environment uses Firebase.

As discussed previously, the method of generating audio signals described in the present disclosure may be used as part of a neuroacoustic feedback treatment in order to treat various neurological and/or mental health conditions. The neuroacoustic treatment method may be adapted or modified for use in treating particular conditions. Some examples of possible therapy applications are outlined below, however other treatment and therapy applications are envisaged.

The neuroacoustic feedback treatment may be used in a therapy to treat PTSD, including complex PTSD. Where the user has experienced a traumatic event or series of events emotional dysregulation is more often than not associated with a trigger. Activation of this trigger can lead to exaggerated emotional responses and extreme physiological reactions. The use of the neuroacoustic feedback treatment with people diagnosed with PTSD can help to rebalance the emotional regulation centre and over time normalise emotional responses and lessen the impact of triggers. In this case, the user would find the stressor frequency that matches their brain function when they are triggered. The disruptor frequency will then break the connection between the trigger and the emotional response. In some examples, the user will listen to the combination of frequencies for 20 minutes. By the end of the session the user should feel calmer and more relaxed and over time the brain will learn to maintain health patterns. Eventually the trigger and associated emotional response will normalise.

The neuroacoustic feedback treatment may be used in a therapy to treat stress. Stress can be defined as any type of change that causes physical, emotional or psychological strain. The use of neuroacoustic feedback treatment in the treatment of stress is substantially similar to the treatment for PTSD.

The neuroacoustic feedback treatment may be used in a therapy to treat generalised anxiety disorder (GAD). Generalised anxiety disorder is where you feel anxious most of the time. Symptoms of generalised anxiety disorder vary from person to person, but include constant worrying, a sense of dread and difficulty concentrating. Where someone suffers from anxiety in a more generalised way (i.e., not associated with a particular trigger), the application may be delivered in a similar way to the above-described treatments for PTSD and stress. In such examples, the application will work to reduce anxiety levels on an everyday basis, rather than in relation to a trigger.

The neuroacoustic feedback treatment may be used in a therapy to treat ADHD. Attention deficit hyperactivity disorder is a condition that includes symptoms such as being restless and having trouble concentrating. Symptoms of attention deficit hyperactivity disorder include a short attention span, constantly fidgeting and acting without thinking. Different types of ADHD may require different methods of apply the neuroacoustic feedback treatment. Theta dominant ADHD will need delivery of tone frequencies that stimulate alpha and beta frequencies in the brain. Beta dominant ADHD needs delivery of tone frequencies that stimulate alpha frequencies.

The neuroacoustic feedback treatment may be used in a therapy to treat bi-polar disorder. Studies of neurocognitive function in bipolar disorder indicate deficits in three core domains: attention, executive function, and emotional processing. Initial research suggests that people brain's who are affected by bi-polar oscillate between beta brain function (during manic phases) and theta brain function (during depressive phases).

The neuroacoustic feedback treatment may be used in a therapy to treat depression. Depression is a low mood that lasts for weeks or months and affects daily life. Symptoms of depression include feeling unhappy or hopeless, low self-esteem and finding no pleasure in things.

The neuroacoustic feedback treatment may be used in a therapy to treat postnatal depression. Postnatal depression is a type of depression that parents can have after having a baby. Symptoms of postnatal depression include constant sadness, lack of energy and difficulty bonding with your baby.

The neuroacoustic feedback treatment may be used in a therapy to treat perinatal mental health issues. Medication for mental health conditions can present complicated decisions for expectant mothers. There is evidence to suggest some forms of medication may be harmful to an unborn child. The neuroacoustic feedback treatment can be used as a treatment solution for pregnant women to maintain a non-harmful treatment regime during pregnancy for the treatment of mental health conditions, including any of the conditions discussed in this disclosure. Such treatments could also continue post birth, for example during breast feeding.

The neuroacoustic feedback treatment may be used in a therapy to treat eating disorders. In some examples, the neuroacoustic feedback treatment can be used to manage triggers using the application. The treatment can also help with binge eating responses caused by emotional dysregulation and other triggers from food in cases of anorexia. Similar treatment options could be used in a treatment for obesity.

The neuroacoustic feedback treatment may be used in a therapy to improve impulse control. Such treatment may be used to treat addiction (including smoking). Such treatment may also be used to treat violent or otherwise dangerous impulses.

The neuroacoustic feedback treatment may be used in a therapy to treat phobias.

The neuroacoustic feedback treatment may be used in a therapy to treat autism. Autism is a lifelong developmental disability which affects how people communicate and interact with the world.

The NAF therapy described above may be administered in conjunction with a suitable pharmaceutical drug therapy selected according to the diagnosis of the underlying health condition, serving to improve clinical outcomes over just administering the drug therapy alone in one or more ways.

1. A computer implemented method for generating digital audio signals for stimulating a user's brain, comprising the steps of:
generating a first audio signal which changes frequency at a first rate for playback to a patient through a first speaker of a stereo headset;
receiving an input which identifies a stressor frequency for the patient;
generating a second audio signal which changes frequency at a second rate for playback to the patient through a second speaker of the stereo headset, while simultaneously playing an audio signal at the stressor frequency through the first speaker of the stereo headset, wherein an initial frequency for the second audio signal is selected to be within a range extending from the stressor frequency to a frequency 40 Hz higher than the stressor frequency;
receiving an input which identifies a disruptor frequency for the user; and,
storing the identified stressor frequency and the identified disruptor frequency for subsequent playback to the user.

2. The method according to claim 1, wherein a frequency range of the first audio signal is between 40 Hz and 1.5 kHz.

3. The method according to claim 1, wherein an initial frequency for the first audio signal is at least 40 Hz and is thereafter increased incrementally at the first rate.

4. The method according to claim 1, wherein an initial frequency for the first audio signal is determined using user data.

5. The method according to claim 1, wherein an initial frequency for the second audio signal is selected to be equal to the stressor frequency and is thereafter increased incrementally at the second rate.

6. The method according to claim 1, wherein the input which identifies the stressor frequency and/or the disruptor frequency is a user input, preferably a touch gesture.

7. The method according to claim 1, wherein the input which identifies the stressor frequency and/or the disruptor frequency is a user biomarker signal.

8. The method according to claim 7, in which the user biomarker signal is monitored heart rate variability (HRV).

9. The method according to claim 1, wherein the stressor frequency is determined by a reduction in an HRV value for the user, and the disruptor frequency is determined by an increase in an HRV value for the user.

10. The method according to claim 1, wherein the first rate is higher than the second rate.

11. The method according to claim 1, wherein the first rate gives rise to a non-linear incremental change in frequency of the first audio signal.

12. The method according to claim 1, in which the first rate increases the frequency of the first audio signal in increments of k×fc, where fc is the current frequency of the first audio signal and k is a constant.

13. The method according to claim 12, wherein k is a value of at least 1, and preferably at least 1.03.

14. The method according to claim 1, wherein the second rate gives rise to linear incremental change in frequency of the second audio signal.

15. The method according to claim 1, in which the second rate increases the frequency of the second audio signal in increments of at least 0.2 Hz per second, and preferably at least 0.5 Hz per second.

16. The method according to claim 1, in which the first audio signal and the second audio signal are simple sine waves with minimal coloration.

17. The method according to claim 1, wherein the headset comprises a set of over-ear headphones, on-ear headphones, in-ear speaker buds, or bone conduction speakers.

18. The method according to claim 1, wherein the first audio signal and the second audio signal are not pre-recorded.

19. A computer program comprising instructions, that when executed, perform the method of claim 1.

20. A digital audio processing device including instructions, that when executed by a processor, are configured to cause the processor to perform the method of claim 1.

21. The device according to claim 20, comprising a smartphone, a smart watch, a tablet computer, a laptop computer, or a personal wellness pod.

22. A method of treatment for a health condition using neuroacoustic feedback (NAF) therapy, comprising the steps of generating a first audio signal at a stressor frequency and a second audio signal at a disruptor frequency for simultaneous playback to a user through respective speakers of a stereo headset, wherein the stressor frequency and the disruptor frequency are determined by the method of claim 1.

23. The method of treatment according to claim 22, wherein the health condition is one of stress, anxiety, post-traumatic stress disorder (PTSD), depression, addiction, attention deficit hyperactivity disorder (ADHD), bipolar affective disorder, phobia, behavioural and emotional disorders with children, eating disorders, autism and obsessive-compulsive disorder (OCD).

24. The method of treatment according to claim 22, further comprising administering a pharmaceutical treatment as a co-therapy for treating the health condition.
